(19) United States
(12) Patent Application Publication
(10) Pub. No.: US 2024/0425802 A1
(43) Pub. Date: Dec. 26, 2024

(54) Lyophilizing Honey with a Buffering Agent

(71) Applicant: Steven Shoup, New Plymouth, ID (US)

(72) Inventor: Steven Shoup, New Plymouth, ID (US)

(21) Appl. No.: 18/465,229

(22) Filed: Sep. 12, 2023

Related U.S. Application Data
(60) Provisional application No. 63/509,292, filed on Jun. 21, 2023.

(51) Int. Cl. C12N 1/04

(52) U.S. Cl. CPC C12N 1/04

(57) ABSTRACT

Compositions and methods are provided for lyophilizing microbes suspended in a medium. Specifically, a food source comprising honey and a buffering medium form a fluffy cake after the lyophilizing.

### CROSS REFERENCE TO RELATED APPLICATION

This application claims priority to U.S. Provisional Application No. 63/509,292, filed Jun. 21, 2023, which is incorporated herein by reference in its entirety.

### BACKGROUND OF THE INVENTION

Growing multiple different microbes together or having them coexist can be problematic. Many microbes are antagonistic to each other or have faster growth rates at certain conditions monopolizing essential nutrients. Thus, incorporating wide spectrum of microbes is simultaneously as challenging as it is desirable.

Naturally, the greater the concentration of the beneficial microbes, the more viable a product becomes. In order to create a high concentration of microbes, it is important to have a food source that is extremely nutritious for the broad array different microbes. The media generally needs to contain sources of carbon, nitrogen and vitamins. Molasses is a widely utilizable carbon source for microbes and is the most commonly used growth media. Nitrogen sources can often include peptone, yeast extract, and malt extract. Again, the problem exists on how to ensure all the different classes of microbes can grow without monopolizing the food source required by the other microbes.

A further problem exists when one type of microbe grows too rapidly. The brew will tend to form a film or a matty layer created by the over-abundance of a particular microbe. These matty layers or matts can be found on the top of the brew, on the bottom, or somewhere in the middle depending on the particular microbe. This matty layer creates a problem during the processing of batch into a marketable product. These matts are difficult to break down and are difficult to filter out of the brew. Consequently, these matts present an issue for creating a water-soluble product.

The industry typically brews the microbes in large batches with molasses. Molasses is an extremely processed product but is relatively inexpensive. It has lots of its sugar content removed during its first boiling and creation of a first syrup. Because of its extreme processing, molasses typically has material that the microbes do not consume and do not dissolve in water. In addition, molasses generally needs additional vitamins and amino acids to be added to grow some of the beneficial microbes. Often, these additions are not water soluble and create post lyophilization issues.

Honey should be considered as a food source since it contains a high concentration of sugars and essential vitamins especially the B vitamins. However, honey has low glass transition temperatures (Tg), which makes freeze drying difficult. During dehydration, honey typically becomes extremely sticky, hard and very difficult to handle as a product. Although honey does not lyophilize well, when used as a food source, the microbial count skyrockets compared to most other food sources.

Because of adverse effects of heat and water, lyophilization has become a standard practice in many industries, including agriculture, biotechnology, and pharmaceutical. Dehydration by lyophilization offers several advantages including improved stability, increased shelf life, and reduced transportation and storage costs. Because lyophilization creates a dry medium encompassing the microbes, many micro-organisms survive well when lyophilized. These microbes can be easily rehydrated and rejuvenated after prolonged periods of time. Thus, lyophilization provides a convenient method for long-term storage of biological products while preserving their activity.

Lyophilization also reduces weight and volume. This reduction reduces shipping costs and environmental impact. Eliminating the need for shipping while maintaining product stability, such as dry ice, further reduce costs. Long transportation distances with hot or humid environments put a significant strain on cold chain transport. With freeze-drying, a temperature-controlled chain is not necessary in most instances.

Thus, there is a need to be able to create a commercially viable dry powder containing lyophilized biological products that used honey as the food source.

### BRIEF SUMMARY OF THE INVENTION

The present invention discloses compositions and methods in various embodiments for lyophilizing microbial isolates after increasing the concentration of the microbes by using honey as a food source.

In one embodiment, a method is disclosed for lyophilizing microbes. This method includes increasing the concentration of the microbes by feeding the microbes a food source comprising of honey, lyophilizing a buffering medium containing the microbes, and the buffering medium forming a fluffy cake after the lyophilizing.

In this embodiment, the lyophilizing has a drying process that removes water above 0 degrees Celsius longer than removal of water below 0 degrees Celsius. The fluffy cake has a texture that is not clumpy and is filterable by an unaided vibration of a 20 mesh shaker screen.

Further disclosed, the food source also includes yeast. In another embodiment, the food source comprises yeast extract. The honey's wax is removed prior to use as a food source. After cooking, the food source is filtered prior to the feeding of the microbes.

The buffering medium disclosed is comprised of a sucrose solution. Preferably, the sucrose solution is obtained from sugar cane. The solution is between a 5% sucrose solution and a 10% sucrose solution inclusive after adding the sucrose solution to the microbes.

In another embodiment, a composition comprising of lyophilized microbes in a medium includes a food source of honey, a buffering medium, and the buffering medium forms a fluffy cake after the lyophilizing.

In this embodiment, the honey is processed to have the honey's wax removed. The food source also has yeast or yeast extract. After cooking, the food source is filtered to 100 microns or finer.

As disclosed, buffering medium is comprised of a sucrose solution. Preferably, the sucrose solution is obtained from sugar cane. the sucrose solution is between a 5% sucrose solution and a 10% sucrose solution inclusive after adding the sucrose solution to the microbes.

Further disclosed, the medium is dried by a lyophilization process that removes water above 0 degrees Celsius longer than below 0 degrees Celsius. This results in a fluffy cake has a texture that is not clumpy and is filterable by an unaided vibration of a 20-mesh shaker screen.

### BRIEF DESCRIPTION OF THE DRAWINGS

Various embodiments are illustrated by way of examples in the figures of the accompanying drawings. Such embodiments are demonstrative and not intended to be exhaustive or exclusive embodiments of the present subject matter. The drawings are not necessarily to scale, emphasis instead being placed on illustrating the principals of the invention.

FIG. 1 is a flow chart depicting the major steps of the preferred embodiment of the present invention.

FIG. 2 depicts the step of cooking a microbial food source.

FIG. 3 depicts the step of brewing a microbial solution.

FIG. 4 depicts the lyophilizing a microbial medium.

FIG. 5 depicts the programed lyophilizing schedule.

FIG. 6 depicts the step of crumbling the microbial medium.

FIG. 7 depicts the step of milling the microbial medium.

FIG. 8 depicts the step of shaking the medium into a blended powder.

### DETAILED DESCRIPTION OF THE INVENTION

The description that follows includes compositions, systems, methods, and apparatuses that embody various elements of the present disclosure. However, it should be understood that the described disclosure may be practiced in a variety of forms in addition to those described herein. Accordingly, the referenced drawings show, by way of illustration, specific embodiments in which the invention may be practiced. It is to be understood that other embodiments may be utilized, and structural changes may be made without departing from the scope of the claims. It is further understood that the steps described with respect to the disclosed processes may be performed in differing order and are not limited to the steps presented herein.

The present invention discloses compositions and methods in various embodiments for enhancing the concentration of microbes with a diversity of classes suspended in a powder after lyophilization. The preferred embodiments are described below.

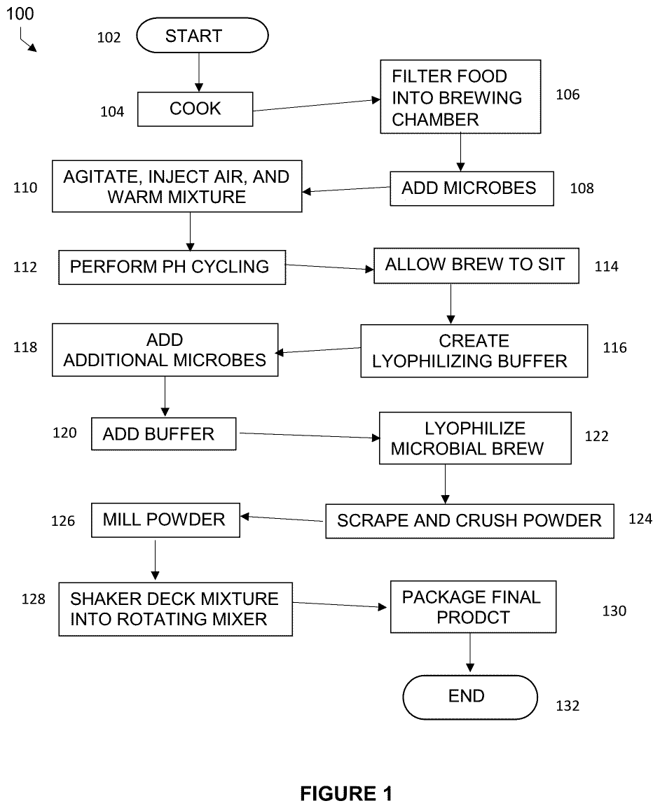

Referring to FIG. 1, illustrated is a flowchart 100 for the major steps for enhancing the concentration of microbes with a diversity of classes suspended in a powder.

Following the start step 102, a food source is cooked as described in step 104.

In step 104, care should be used to choose foods and components that are 100% water soluble and stay in suspension to help ensure water solubility of the final product.

Honey is used as a food source since it contains a high concentration of sugars and essential vitamins especially the B vitamins. Start with 50% brewer's yeast by weight and 50% processed honey by weight. The yeast selected should be 100% water soluble. Processed honey with the waxes removes is used to help ensure solubility. The industry typically uses around 10% yeast; however, a 50% yeast mixture increases the yield when combined with honey. To ensure consistency, the honey should be a blend that uses a large variety of honeys from multiple sources.

In step 106, the food source is filtered and added into a brewing chamber. Cool the food mixture to below 95 degrees. The cooked mixture is then filtered and cleaned down to at least 10 microns before going into a brewing vessel. The filtering removes most of the non-soluble material in the solution including the yeast cell wall debris. The resultant is approximately 39 to 40 liters of food to be added to the brewing vessel.

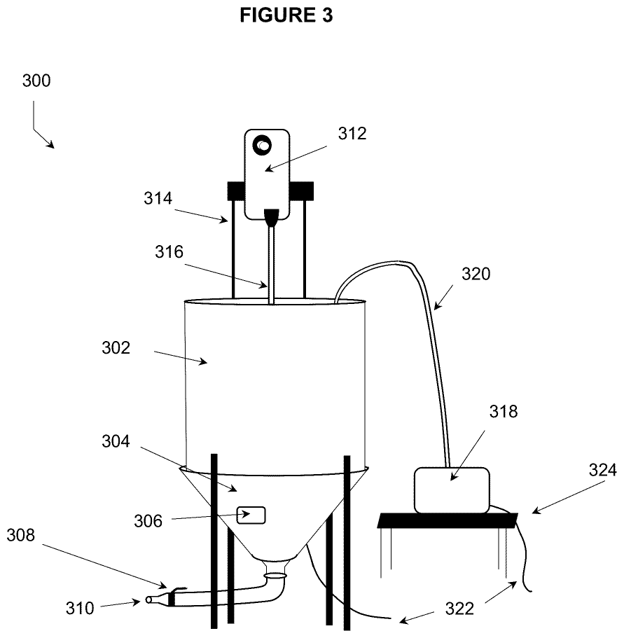

In step 108, after adding the food mixture, 8 liters of microbes from a previous batch is added to the feeding tank. It is preferred to have a broad spectrum of the microbial isolates. Rhizobacteria can suppress pathogen ingress into the plant roots and are essential for biological nitrogen fixation. Actinomycetes produce powerful antibiotic compounds and eradicate a large array of soil borne pathogens and nematodes. Other bacterial isolates can accelerate the microbial processes that augment the availability of nutrients in a form easy to assimilate by plants. Similarly, fungi are organisms that can secrete growth promoting hormones. In addition, saprophytic fungi can produce secondary metabolites that are active in suppressing soil borne pathogens and plant parasitic nematodes. Endomycorrhizae live in the root tissue and are powerful growth stimulators. Ectomycorrhizae form a sheath around plant roots preventing nematodes and pathogen penetration as well as promote nutrient absorption. By using the disclosed pH cycling, the concentration of these microbes is greatly increased and are brewed together. The step of brewing 110 of the microbial solution is depicted in reference to FIG. 3.

In step 110, agitation, warming, and air injection is started after adding the microbes. Compressed and dried air is injected into the bottom of the feeding tank. Direct oxygen can be detrimental. A light bubble similar to a fish tank is utilized just to keep brew oxygenated. The stirring rotates at 45 to 70 rpm to keep the mixture moving and lightly agitated. External heating pads surrounding the vessel are set to keep the mixture to around 85-95 degrees.

In step 112, after adding the microbes, the resultant mixture is around 6.3 pH. In order to maximize the growth of a wide diversity of microbes with the temperature held constant, the pH needs to be managed such that all the microbes grow without any one microbe severely dominating. Accordingly, during the growing process, the brew is controlled to a range of pH of around 6 to a pH in the mid 4s. Since different microbes thrive at differing pHs, cycling the pH ranges allows the microbes to grow together to feed and multiply without other microbes dominating. If the brew is allowed to sit too long in the low 4s pH, a resultant acidophilic actinomycetes bloom occurs. The cycling of the pHs allows the microbes to grow without any one particular microbe dominating and eliminates the matting layer issue.

Mined sodium carbonate that is completely water soluble is preferred to be used to alter the pH. Use 5% mixture of sodium carbonate to water. When the brew is in the mid 4s pH, add sodium carbonate while continuing to agitate. The first pH crash to mid 4s pH usually takes around 5 to 5.5 hours and will need about 2 liters of the sodium carbonate to raise the batch to around a 6 pH. The second crash to the mid 4s PH usually takes less time of 3 to 4 hours and the addition of around 3 liters to raise the pH back to around a pH of 6. The third crash may take as long as 13 hours. Typically, the total of 8.5 to 9 liters of the sodium carbonate is used for the 3 cycles.

Running the brew through two pH cycles greatly increases the microbial yields of all the differing microbes. A third cycle is preferred to be performed to further increase microbial count. At the end of the pH brew cycling, the brew will be at approximately a 6 ph.

In step 114, allow the brew to sit for another 48 hours in order to maximize count. As the brew sits, the brew will creep up into 7s pH. Some of microbes will diminish if the brew hits an 8 pH value.

In step 116, the lyophilizing buffer or excipient is created. In a large mix vessel, add 3200 grams of cane sugar to water until the total volume is 4 liters. Mix until the cane sugar is fully dissolved. Ideally, the mixture is concentrated to act as a buffer with the cane sugar staying suspended at room temperature. In order to make the product rise as a airy cake, an 8% mixture after adding the sucrose solution to the microbes is used. Over a 10% concentration will start killing microbes as the sucrose is toxic to some of the microbes. Under 5%, the end product is sticky and gooey and is difficult to create a water-soluble product.

In step 118, any additional microbes are added to the microbial brew. If endo-mycorrhizae are to be added to brew, it should be added at this time. Endo-mycorrhizae may take up to 10 days to brew. Most endo-mycorrhizae are grown on plant roots and harvested by scraping or crushing the roots. Endo-mycorrhizae grown this way make the product non-water soluble. However, certain foods can allow endo-mycorrhizae to grow using the methodology described in this invention and can be water soluble.

In step 120, add the sugar solution to brew containing the microbes. Turn the agitator up and slowly add the sugar buffer solution. The lyoprotectant used in this embodiment of the invention is sucrose, which is a glucose linked via its Cl carbon to fructose. By using honey and sucrose as a dehydration buffer, the final product becomes a fluffy cake with a fine texture and excellent solubility characteristics. If there is not enough sugar, the end product is a sticky mass that clumps because of the honey.

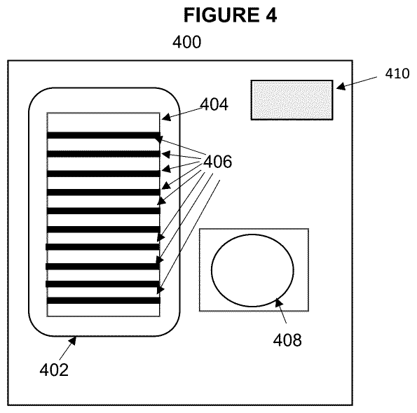

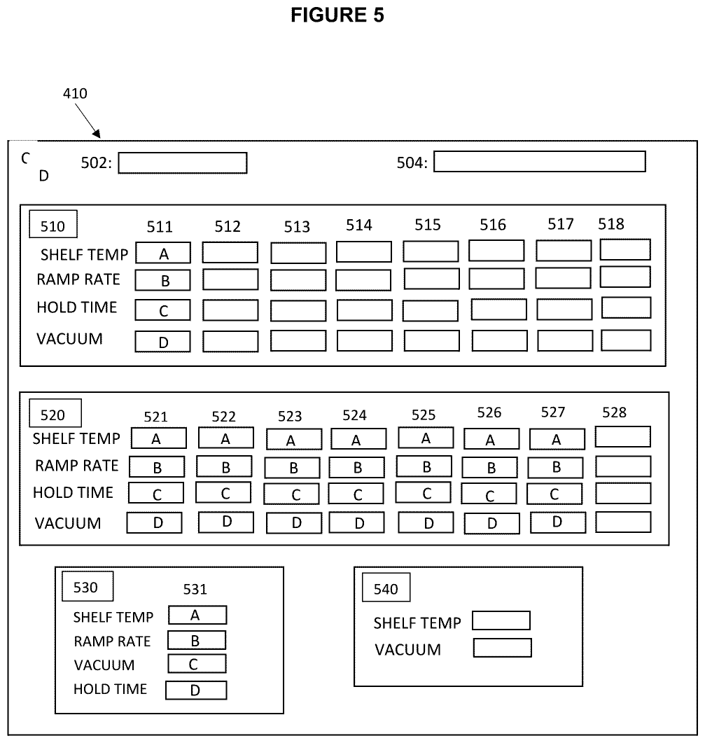

In step 122, the microbial medium is lyophilized as depicted in reference to FIG. 4. Pour the buffered solution into trays for lyophilization. In this preferred embodiment, 2100 ml is poured into a tray and then set the programming as referenced in FIG. 5. Lyophilize the solution for approximately 60 hours to 70 hours. Preserving microbes by lyophilization necessitates that the microbes are suspended in a medium that helps to maintain their viability through freezing, water removal, and subsequent storage. The ideal solution will have a component that helps to form a solid “airy or fluffy cake” which gives body to the bacterial suspension once freeze dried. This process results in a cake that has a texture that is not clumpy and is filterable by an unaided vibration of a 20-mesh shaker screen. Unaided vibration means the cake is placed on the screen and only gravity acts on the cake on the vibrating screen.

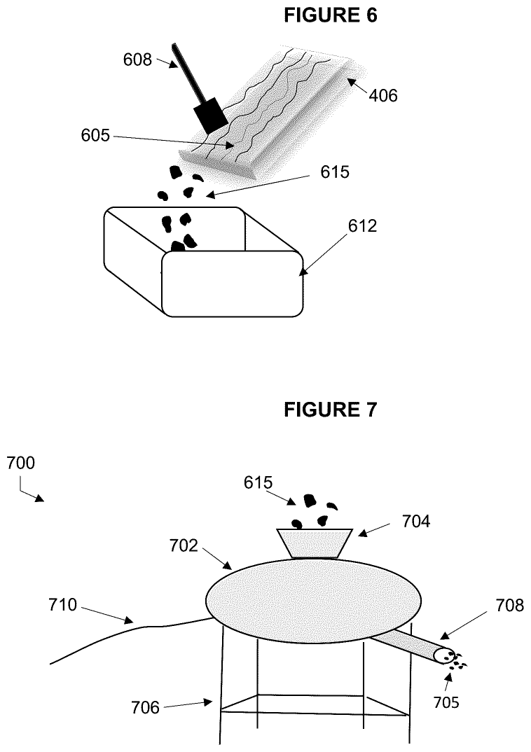

In step 124, after lyophilizing, the microbial medium is removed from the trays and placed into a tub as depicted in reference to FIG. 6. After removing the microbial medium from the lyophilizer, the environment should be controlled to be less than 75° F. and less than 33% humidity. Outside of those parameters, the medium tends to absorb water, and thus, creating a fine powder is extremely difficult. Using a mallet, gently crush the microbial medium into crumbs. In the preferred embodiment, the microbial medium has an airy cake texture and easily obtains a crumbly consistency with slight pressure.

In step 126, a brewer's grain mill crushes the microbial as depicted in reference to FIG. 7. In the preferred embodiment, the brewer's mill uses two stainless steel slow rollers gapped at approximately one millimeter. The microbial medium leaves the brewer's mill as a coarse powder.

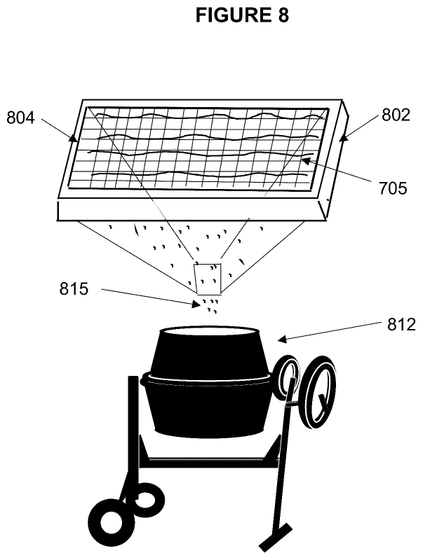

In step 128, the microbial is reduced to a fine powder as depicted in reference to FIG. 8. Shake the coarse microbial powder on a shaker screen using a 20-mesh to 200-mesh screen to create a fine powder. A bulking agent is also added to the shaker screen. The bulking agent preferred is dextrose or sucrose although talc and many other bulking agents can be substituted. The resulting mixed fine powder enters into a horizontal cement mixer to ensure the powder stays moving.

In step 130, the resulting microbial powder is packaged. Seal the powder in an air-tight package. The product is ready for sale, which ends the process as shown in step 132.

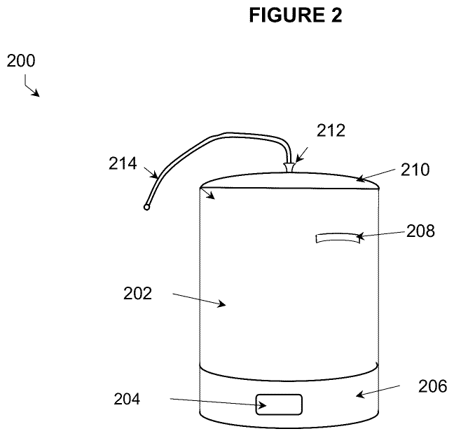

Referring to FIG. 2, the step of cooking 104 the microbial food source is depicted. In the preferred embodiment, 1000 grams of honey is mixed with 1000 grams of brewer's yeast in a commercial cooker 200. The commercial cooker 200 has a cooking chamber 202 with a handle 208, a lid 210, a relief valve 212 with attached tubing 214, and a heating element casing 206 with a heater control panel 204. Water is added until there is 42 liters of mixture is in the cooker 200. The mixture is cooked at a simmer for 20 minutes. This cooking kills any live unwanted bacteria in the water and breaks open the cells of the yeast to create a yeast extract. The cooking temperature should not be hot enough to denature the proteins.

Referring to FIG. 3, depicted is the step of brewing 108 of the microbial solution in a brewing vessel assembly 300. The brewing vessel 302 is an insulated stainless steel cone bottom tank. A nozzle valve 308 at the bottom of the brewing assembly 302 enables the solution to be withdrawn after brewing via the exit nozzle 310.

Agitation is provided by an agitator rotation rod 316 supported by the agitator support stand 314. The agitator controller 312 rotates the agitator rotation rod 316 speed at 45 to 70 rpm to keep the mixture moving and lightly agitated.

Air injection at bottom of the brewing vessel 302 is started after adding the microbes. Compressed and dried air is run through medical grade air filtration system 318 before injection into the feeding tank 302 by an air tube 320. The air filtration system 318 rests on a small table 324. Direct oxygen can be detrimental. A light bubble similar to a fish tank is utilized just to keep brew oxygenated. Power is supplied to the air filtration system 318 by a power cord 322.

A heating control panel 306 governs the external heating pad 304 that surround the bottom conical section of the vessel 302. The heating pad 304 is set to 85 to 95° F. warm, which heats the mixture to around 85-90 degrees. Power to the vessel assembly 300 is supplied via the power cords 322.

In order to maximize the growth of a wide diversity of microbes with the temperature held constant, the pH needs to be managed such that all the microbes grow without any one microbe severely dominating. Running the brew through two pH cycles greatly increases the microbial yields of all the differing microbes. A third cycle is preferred to be performed to further increase microbial count.

Referring to FIG. 4, depicted is the step of the lyophilizing 122 the microbial medium. The microbial medium is poured into trays 406 and placed on the shelves 404 in the vacuum chamber 402 of the lyophilizer 400 for dehydration. A motor (not shown) is in place to ensure the vacuum chamber 402 is vacuum-tight when closed. The condenser 408 consists of refrigerated coils or plates. During the drying process, ice moves from the frozen product to the condenser by sublimation. A programmable lyophilization screen 410 sets the programming. The programing is disclosed further in reference to FIG. 5. Lyophilize the solution for approximately 60 hours to 70 hours.

Referring to FIG. 5, depicted is the programmable lyophilization screen 410. The programmable lyophilization screen 410 has and batch reference input 502 and a product input 504. This lyophilization screen 410 has within it a freeze settings input screen 510, a primary drying settings input screen 520, a secondary drying input screen 530, and a storage setting input screen 540.

The freezing schedule in set by the freezing input screen 510. In this embodiment, the freezing is preferably accomplished as quick as possible. The first row as show sets the shelf temperatures for the freezing stage. Input 511A is set to −50° Celsius. The second row sets the ramp rate. Input 511B is set to 0 degrees Celsius/minute. The third row sets the hold time. Input 511C is set to 120 minutes. The fourth row sets the vacuum pressure. Input 511D is set to between 100 and 200 mTorr. The other columns of inputs 512 thru 518 are not used. In this embodiment, the freezing of the microbial medium can be accomplished quickly using these settings.

The primary drying stage schedule is set by the primary drying input screen 520. The last column of inputs 528 is not used. The first row as show sets the shelf temperatures in Celsius for the primary drying stage. Input 521A is set to −40°, input 522A is set to −30°, input 523A is set to −20°, input 524A is set to −10°, input 525A is set to 0°, input 526A is set to 10°, and input 527A is set to 20º Celsius. The second row sets the ramp rate. Inputs 521B thru 527B are all set to around 0.2 degrees Celsius/minute. The third row sets the hold time. Input 521C is set to 120 minutes. Inputs 522C thru 526C are set to 180 minutes. Input 527C is set to 120 minutes. The fourth row sets the vacuum pressure. Inputs 521D thru 527D are set to over 150 mTorr.

The secondary drying schedule in set by the secondary drying schedule input screen 530. The first row as show sets the shelf temperatures for the secondary drying stage. Input 531A is set to 25 Celsius. The second row sets the ramp rate. Input 531B is set to 0 degrees Celsius/minute. The third row sets the hold time. Input 531C is set to 9,999 minutes. The fourth row sets the vacuum pressure. Input 511D is set to over 150 mTorr. The other columns of inputs 512 thru 518 are not used. In this embodiment, the secondary drying schedule hold time is set to 9,999 minutes. However, anywhere between 30 to 40 hours is used in practice depending on the convenience of the work schedule of the employees. Accordingly, the storage setting input screen is unnecessary and not utilized.

Referring to FIG. 6, depicted is the step of transferring 124 the post lyophilized microbial medium 605 from the lyophilization tray 406 into a container 612. The microbial medium 605 ideally has an airy cake texture that easily obtains a crumbly consistency with slight pressure. In this embodiment, a spatula 608 is used to scrape the post lyophilized medium 605 from the tray 406. Gently crush the post microbial medium 605 into crumbs 615. The crumbs 615 are not clumpy and are filterable by a vibration of an unaided US 20-mesh shaker screen.

Referring to FIG. 7, the step of pouring 126 the crumbs 615 into a brewer's grain mill 700 is depicted. The brewer's mill 700 is supported by a stand 706 and powered by a power cord 710. The microbial medium crumbs 615 are poured into the mill's funnel 704. In this embodiment, the brewer's mill 700 uses two stainless steel slow rollers (not shown) inside the mill's roller bin 702 gapped at approximately one millimeter. The microbial medium crumbs 615 leaves the brewer's mill by the exit tube 708 as a coarse powder 705.

Referring to FIG. 8, the step of shaking 128 the coarse microbial medium 705 using a shaker box 802 is depicted. Place the coarse microbial powder 705 on a shaker screen 804 using a 20-mesh to 200-mesh screen to create the fine microbial powder 815. A bulking agent is also placed on the shaker scree. The preferred bulking agent is dextrose or sucrose although talc and many other bulking agents can be substituted. On the shaker deck 804, ball bearings (not shown) gently push the coarse microbial powder 705 down on the screen 804. An assortment of light-weight nylon balls ranging from a quarter (¼) inch diameter to a one (1) inch diameter is preferred. The fine microbial powder 815 is shaken into a horizontal cement mixer 812 to ensure that the fine microbial powder 815 and the bulking agent stays moving until packaging.

1. A method of lyophilizing microbes comprising: increasing a concentration of the microbes by feeding the microbes a food source, wherein the food source comprises honey, lyophilizing a buffering medium containing the microbes, and the buffering medium forming a airy cake after the lyophilizing.

2. The method of claim 1, wherein the food source further comprises yeast.

3. The method of claim 1, wherein the honey is processed to have the honey's wax removed.

4. The method of claim 1, further comprising filtering the food source prior to the feeding of the microbes.

5. The method of claim 1, wherein the buffering medium is comprised of a sucrose solution.

6. The method of claim 5, wherein the sucrose solution is obtained from sugar cane.

7. The medium of claim 5, wherein the sucrose solution is between a 5 percent sucrose solution and a 10 percent sucrose solution inclusive after adding the sucrose solution to the microbes.

8. The method of claim 1, wherein the airy cake has a texture that is not clumpy and is filterable by an unaided vibration of a 20 mesh shaker screen.

9. The method of claim 1, wherein the food source comprises yeast extract.

10. The method of claim 1, the lyophilizing process comprises a drying process that removes water above 0 degrees Celsius longer than removal of water below 0 degrees Celsius.

11. A composition comprising of lyophilized microbes in a medium comprising: a food source, wherein the food source comprises honey, a buffering medium, and the buffering medium forming an airy cake after the lyophilizing.

12. The method of claim 11, wherein the food source further comprises yeast.

13. The method of claim 11, wherein the honey is processed to have the honey's wax removed.

14. The method of claim 11, further wherein the food source is filtered to 100 microns or finer.

15. The method of claim 11, wherein the buffering medium is comprised of a sucrose solution.

16. The method of claim 15, wherein the sucrose solution is obtained from sugar cane.

17. The medium of claim 15, wherein the sucrose solution is between a 5% sucrose solution and a 10% sucrose solution inclusive after adding the sucrose solution to the microbes.

18. The method of claim 11, wherein the airy cake has a texture that is not clumpy and is filterable by an unaided vibration of a 20 mesh shaker screen.

19. The method of claim 11, wherein the food source comprises yeast extract.

20. The method of claim 11, wherein the medium is dried by a lyophilization process that removes water above 0 degrees Celsius longer than below 0 degrees Celsius.
